(19) United States
(12) Patent Application Publication
(10) Pub. No.: US 2026/0174373 A1
(43) Pub. Date: Jun. 25, 2026

(54) BIOLOGICAL INFORMATION MEASUREMENT DEVICE

(71) Applicant: OMRON HEALTHCARE Co., Ltd., Kyoto (JP)

(72) Inventors: Tomoyuki NISHIDA, Kyoto (JP); Kenji FUJII, Kyoto (JP); Seiji FUKUNAGA, Kyoto (JP)

(21) Appl. No.: 19/545,658

(22) Filed: Feb. 20, 2026

Related U.S. Application Data
(63) Continuation of application No. PCT/JP2024/040417, filed on Nov. 14, 2024.

(30) Foreign Application Priority Data
Feb. 28, 2024 (JP) 2024-028792

(51) Int. Cl. A61B 5/282; A61B 5/00; A61B 5/256

(52) U.S. Cl. CPC A61B 5/282; A61B 5/256; A61B 5/6831; A61B 2560/0468; A61B 2562/043; A61B 2562/227

(57) ABSTRACT

A biological information measurement device having a main body housing, a band portion in which a plurality of electrodes are disposed and which is used to attach the main body housing to a human body, and a detachable band cover formed of a non-conductive material and configured to be able to cover at least a contact surface of the band portion on a side that comes in contact with a human body. The electrodes each include a protrusion portion provided to protrude from the contact surface of the band portion. The band cover includes a plurality of opening portions, each of the opening portions being through which the protrusion portion of the electrode is provided to protrude. The electrode and the opening portion are configured such that the opening portion is covered by the protrusion portion of the electrode when the band portion is covered by the band cover.

### CROSS-REFERENCE TO RELATED APPLICATIONS

This application is the U.S. national stage application filed pursuant to 35 U.S.C. 365(c) and 120 as a continuation of International Patent Application No. PCT/JP2024/040417, filed Nov. 14, 2024, which application claims priority to Japanese Patent Application No. 2024-028792, filed Feb. 28, 2024, which applications are incorporated herein by reference in their entireties.

### FIELD

The present invention belongs to the technical field related to healthcare, and particularly relates to a biological information measurement device.

### BACKGROUND ART

In the related art, it has become common for an individual to measure information (hereinafter, also referred to as biological information) related to a body and health of the individual such as a blood pressure value and an electrocardiographic waveform on a daily basis by himself/herself by using a measurement device and to utilize the measurement result for health management. Specifically, in recent years, there has been a growing need for early detection of a disease and appropriate treatment performed by constantly wearing a measurement device on the body in daily life and continuously acquiring biological information, and many devices meeting such a need have been proposed (for example, Patent Document 1 or the like).

Patent Document 1 discloses a wearable biological information measurement device that includes an electrocardiographic electrode and that can measure an electrocardiographic waveform. The biological information measurement device described in Patent Document 1 includes a band (belt) having a band shape and extending along a longitudinal direction (circumferential direction of a wrist) and a main body portion provided on the band, and has a configuration in which a plurality of electrodes are disposed in each of the band and the main body portion. This makes it possible to constantly detect biological information such as a heart rate or an electrocardiographic signal (during operation of the device) by simply attaching the device to one arm.

### CITATION LIST—PATENT LITERATURE

Patent Document 1: JP 2021-141955 A

### SUMMARY OF INVENTION

### Technical Problem

In a structure in which an electrode that comes in contact with the skin of a living body is held on the surface of the band as in the device described in Patent Document 1, when the device is worn for a long time, stuffiness or rash is likely to occur due to a substance (sweat, keratin, or the like) derived from the living body. In addition, since the electrodes are disposed on the band, the band cannot be easily washed, which creates a problem from the viewpoint of wearing comfort (and hygiene).

In view of the problems described above, an object of the present invention is to provide a technique that can improve wearability and maintainability of a wearable biological information measurement device including electrodes on a band portion.

### Solution to Problem

A biological information measurement device according to the present invention adopts the following configurations in order to solve the above problems. In other words, the biological information measurement device is a biological information measurement device to be used by being attached to a human body, the biological information measurement device including: a main body housing; a band portion in which a plurality of electrodes are disposed and which is used to attach the main body housing to the human body; and a band cover that is detachable, formed of a non-conductive material, and configured to be able to cover at least a contact surface of the band portion on a side that comes in contact with the human body, wherein the electrodes each include a protrusion portion provided to protrude from the contact surface of the band portion, the band cover includes a plurality of opening portions, each of the opening portions being through which the protrusion portion of the electrode is provided to protrude, and the electrode and the opening portion are configured such that the opening portion is covered by the protrusion portion of the electrode when the band portion is covered by the band cover.

With the above configuration, cleanliness of a portion that always comes in contact with the living body can be easily maintained by replacing the band cover. Further, since cleaning can be completed only by attaching and detaching the band cover, maintainability of the device is improved, and the operating time (measurement time of biological information) of the device can be increased.

Furthermore, since the surface of the skin comes in contact with the band cover when the band cover is used, by devising the material of the band cover (that is, regardless of the material of the band), stuffiness or rash when the device is attached can be prevented. In addition, since the band cover can be designed in various ways, the cover can be easily replaced in accordance with the time, place, and occasion.

Further, the electrode may be configured such that the protrusion portion is attachable to and detachable from the band portion. Specifically, for example, the electrode may be provided with a base portion connected to a signal line in the band, and the protrusion portion configured to protrude from the contact surface between the band and the skin of the living body may be fitted to the base portion in manner of a so-called snap button so as to be detachable. Alternatively, at least one of the protrusion portion or the base portion of the electrode may be formed of a magnet, and the protrusion portion and the base portion may be configured to be attachable and detachable by being magnetically attracted to each other. With such a configuration, not only the band cover but also the electrode can be replaced, and thus the maintainability of the device can be further improved.

Furthermore, when the band portion is covered by the band cover, the band cover may be fixed by sandwiching at least a peripheral edge portion of the opening portion between the protrusion portion of the electrode and the contact surface. In other words, by sandwiching the band cover between the contact surface of the band and the protrusion portion (an end portion on the contact surface side) of the electrode, the position of the opening portion of the band cover may be prevented from being shifted.

With such a configuration, the protrusion portion of the electrode can be assembled after the band cover is attached to the band, and thus the attachment can be easily performed, and the protrusion portion is assembled in this manner, and thus the position of the opening portion of the band cover can also be prevented from being shifted.

In addition, the band cover may be formed of a material having elasticity, and the electrode and the opening portion may be configured such that the opening portion is covered by the protrusion portion of the electrode even when the opening portion is maximally expanded with the elasticity.

In a case where the band cover is made of a material having elasticity, the opening portion is expanded by applying pulling force to the band cover. In this case, if the opening portion is expanded to be larger than the diameter of the protrusion portion of the electrode, the position of the opening portion of the band cover is likely to be shifted to cover the electrode. In this regard, by reducing the size of the opening portion, using a material having low elasticity, or the like, the opening portion is configured to have a diameter smaller than the diameter of the protrusion portion of the electrode even when the opening portion is maximally expanded. As a result, the electrode can be prevented from being detached from the opening portion even when the peripheral edge portion of the opening portion is not pressed by the electrode.

Moreover, the protrusion portion of the electrode may be provided with an elastic member at a base end portion of an outer periphery in a plan view, and when the band portion is covered by the band cover, the band cover may be fixed by restricting movement of a peripheral edge portion of the opening portion by friction with the elastic member. The elastic member may be made of resin such as rubber.

With such a configuration, even when the force of sandwiching the band cover between the protrusion portion of the electrode and the contact surface of the band is weak, the position of the opening portion of the band cover can be prevented from being shifted by frictional force of the elastic member.

Further, the band portion may be provided with a plurality of conductive fitting portions each fitted to the protrusion portion, and the protrusion portion of the electrode may be attachable to and detachable from the fitting portion. A plurality of signal lines each electrically connecting a corresponding one of the fitting portions and the main body housing may be disposed inside the band portion to avoid the other fitting portions. In other words, the electrode in the band may be formed of the fitting portion disposed in the band and the protrusion portion. With such a configuration, the protrusion portions (that is, a portion that comes in contact with the living body) of the plurality of electrodes provided on the band can be easily replaced, and high maintainability can be obtained. Additionally, at least the fitting portion may have magnetic force, and the fitting portion and the protrusion portion may be fixed to each other by the magnetic force.

Furthermore, the protrusion portion of the electrode may be configured to be attachable to and detachable from any of the plurality of fitting portions, and the arrangement and/or number of the electrodes may be configured to be changeable by providing the fitting portion to which the protrusion portion is not fitted. In other words, the band may be provided with a larger number of fitting portions than the number of electrodes actually used for measurement of biological information. With such a configuration, the arrangement of the electrodes can be easily changed according to individual differences in the size of an attaching site (for example, the circumference of the arm), and thus the device having high convenience can be provided. In addition, by using the band cover in which after the arrangement of the electrodes is determined, the position of the protrusion portion of the electrode and the position of the opening portion are matched, the fitting portion to which the protrusion portion is not fitted can be covered by the band cover.

Further, the fitting portion may be configured to be slidable, and the arrangement of the electrode may be configured to be changeable by sliding the fitting portion. Even with such a configuration, the arrangement of the electrode can be easily changed.

Furthermore, the band cover may be provided with an adhesive surface on a side that comes in contact with the contact surface. With such a configuration, the band cover does not need to be formed in a cylindrical shape or a bag shape so as to cover both surfaces of the band, and the band cover can be used by being attached only to the contact surface side. In addition, the adhesive surface is formed of a gel sheet or the like so as to be washable and thus can be used repeatedly.

Note that the configurations described above can be combined with one another to constitute the present invention unless the combination leads to technical contradiction.

### Advantageous Effects of Invention

According to the present invention, in a wearable biological information measurement device including an electrode on a band portion, the technique that can improve wearability and maintainability of the device can be provided.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is an external perspective view illustrating an outline of a biological information measurement device according to an embodiment of the present invention.

FIG. 2(A) is an external perspective view illustrating the biological information measurement device according to the embodiment from which a band cover is removed. FIG. 2(B) is a diagram illustrating an outline of an inner circumferential surface of a band portion of the biological information measurement device according to the embodiment.

FIG. 3(A) is a first schematic diagram illustrating a structure of an electrode and a mode of arrangement of the electrode on a fixing band according to the embodiment. FIG. 3(B) is a second schematic diagram illustrating the structure of the electrode and a mode of arrangement of the electrode on the fixing band according to the embodiment.

FIG. 4(A) is a first schematic plan view illustrating a configuration of the band cover according to the embodiment. FIG. 4(B) is a second schematic plan view illustrating the configuration of the band cover according to the embodiment.

FIG. 5 is a block diagram illustrating a functional configuration of the biological information measurement device according to the example.

FIG. 6(A) is a schematic diagram illustrating an arrangement relationship between the electrode and the band cover according to a first modified example of the embodiment.

FIG. 6(B) is a schematic diagram illustrating an arrangement relationship between the electrode and the band cover according to a second modified example of the embodiment.

FIG. 7 is a schematic diagram illustrating a configuration of an electrode according to a third modified example of the embodiment.

FIG. 8(A) is a schematic diagram illustrating a configuration of a band portion and electrodes according to a fourth modified example of the embodiment.

FIG. 8(B) is a schematic diagram illustrating a configuration of a band portion and an electrode according to a fifth modified example of the embodiment.

### DESCRIPTION OF EMBODIMENTS

### Embodiments

Embodiments of the present invention will be specifically described below with reference to the drawings. Note that unless otherwise specified, the dimensions, materials, shapes, relative arrangements, and the like of the configurations described in the following embodiments are not intended to limit the scope of the present invention to those alone.

### Overall Configuration of Device

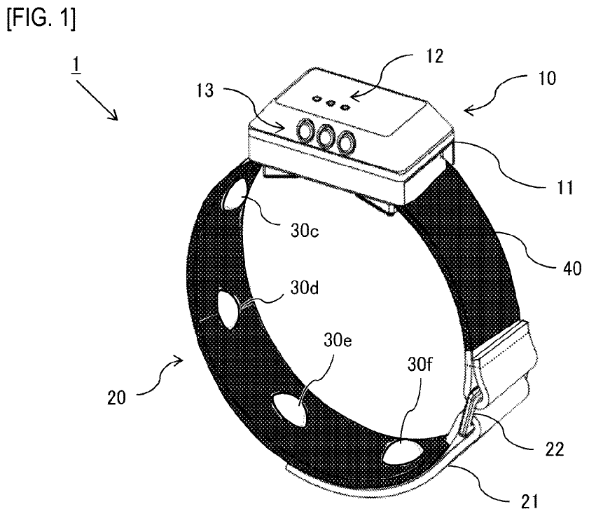

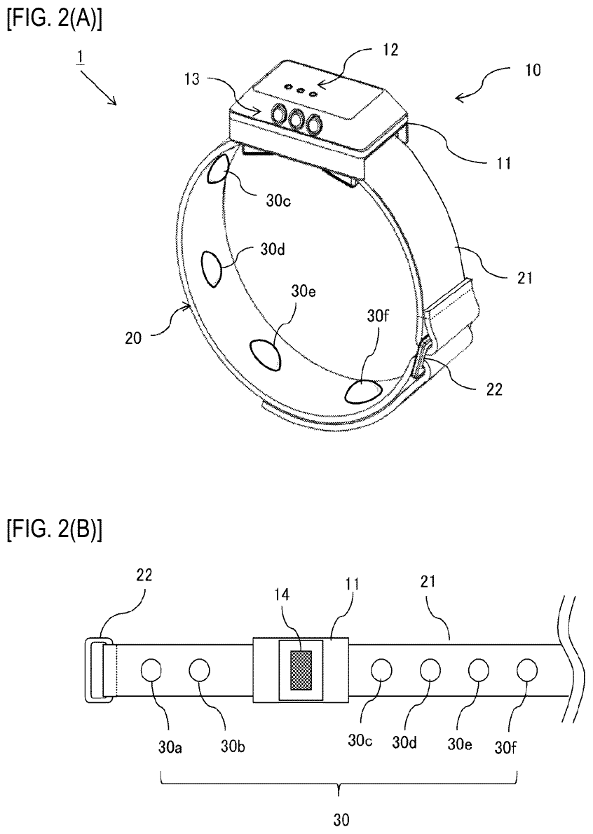

FIG. 1, FIG. 2(A), and FIG. 2(B) are schematic diagrams illustrating a configuration of a biological information measurement device 1 in a first embodiment. FIG. 1A illustrates an external perspective view of the biological information measurement device 1, FIG. 2(A) illustrates an external perspective view of the biological information measurement device 1 from which a band cover 40 is removed, and FIG. 2(B) illustrates an outline of an inner circumferential surface (a surface that comes in contact with the skin surface) of a band portion 20 of the biological information measurement device 1 from which the band cover is removed likewise (hereinafter, the surface is also referred to as “contact surface”).

As illustrated in FIG. 1, FIG. 2(A), and FIG. 2(B), the biological information measurement device 1 generally includes a main body portion 10 including a main body housing 11, an LED indicator 12, an operation button 13, a pulse wave sensor 14, a control unit 110, and the like, a band portion 20 including a fixing band 21, a band annular loop 22, and a plurality of electrodes 30a, 30b, 30c, 30d, 30e, and 30f, and a band cover 40 configured to be attachable to and detachable from the band portion 20.

Additionally, although not illustrated, the fixing band 21 is provided with a hook-and-loop fastener portion, and one end portion of the fixing band 21 is passed through the band annular loop 22 and folded back to be engaged with the hook-and-loop fastener. Thus, the fixing band 21 is formed into a ring shape and can be fixed to an attaching site.

### Configuration of Electrode

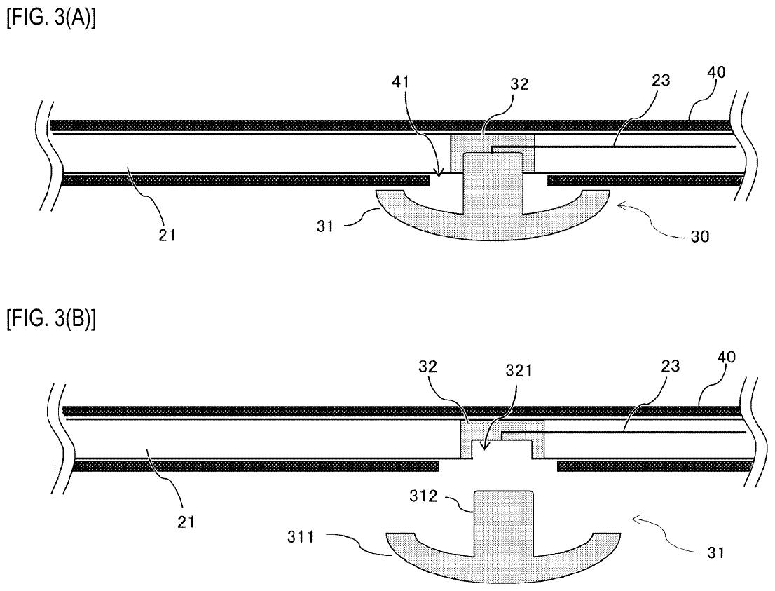

Next, the configuration of the electrodes 30a, 30b, 30c, 30d, 30e, and 30f of the biological information measurement device 1 according to the present embodiment will be described with reference to FIG. 3(A) and FIG. 3(B). Additionally, in the following description, the electrodes will be referred to as electrodes 30 unless the electrodes need to be described while being distinguished from each other. FIG. 3(A) and FIG. 3(B) are schematic diagrams illustrating a structure of the electrode 30 and a mode of arrangement of the electrode 30 on the fixing band 21.

As illustrated in FIGS. 3(A) and 3(B), each of the electrodes 30 is configured to include a protrusion portion 31 that protrudes in a dome shape on the contact surface side of the fixing band 21, and a fitting portion 32 that is disposed into the fixing band 21. Further, each electrode 30 is made of a conductive material (e.g., stainless steel) and is electrically connected to the main body portion 10 via a conductive wire 23 disposed inside the fixing band 21. A user can acquire an electrocardiographic signal via each electrode 30 by wearing the biological information measurement device 1 on, for example, the left upper arm with the use of the fixing band 21 such that the protrusion portion 31 of each electrode 30 is in contact with the skin surface.

Furthermore, as illustrated in FIG. 3(B), the protrusion portion 31 of the electrode 30 is composed of an umbrella portion 311 formed in a dome shape and a shaft portion 312 extending in a cylindrical shape from a central portion of the umbrella portion 311. A fitting recessed portion 321 to which the shaft portion 312 is fitted is formed in the fitting portion 32. Thus, the protrusion portion 31 of the electrode 30 is detachably fitted to the fitting portion 32 so as to be like a so-called snap button. Note that FIG. 3(A) illustrates a state where the protrusion portion 31 and the fitting portion 32 are fitted (a fitted state), and FIG. 3(B) illustrates a state where fitting of the protrusion portion 31 to the fitting portion 32 is released (a released state).

### Configuration of Band Cover

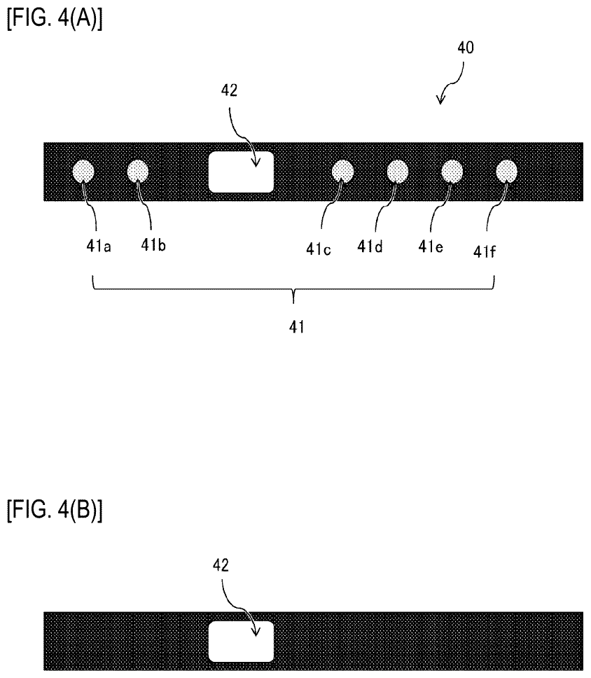

Next, the configuration of the band cover 40 will be described with reference to FIG. 4(A) and FIG. 4(B). FIG. 4(A) is a schematic plan view of the band cover 40 as viewed from the contact surface side, and FIG. 4(B) is a schematic plan view of the band cover 40 as viewed from the upper surface side. As illustrated in FIG. 4(A) and FIG. 4(B), the band cover 40 has a substantially rectangular shape having a longitudinal direction in a plan view, and is formed in a flat tubular shape with both ends in the longitudinal direction open. In addition, the band cover 40 is formed of a non-conductive cloth material having elasticity, but a forming method thereof is not particularly limited. The band cover 40 may be formed by knitting, or may be formed by joining a plurality of cloth materials by sewing, thermal welding, or the like.

Further, as illustrated in FIG. 4(A), the band cover 40 is provided with a plurality of electrode openings 41a, 41b, 41c, 41d, 41e, and 41f for exposing the electrodes 30 and a main body opening 42 for exposing the main body housing 11 when the band cover 40 is attached to the band portion 20. In the following description, the electrode openings 41a, 41b, 41c, 41d, 41e, and 41f are referred to as electrode openings 41 unless the electrode openings need to be described while being distinguished from each another. Each of the electrode openings 41 is provided only on the contact surface side of the band cover 40, and the main body opening 42 is provided in any portion on the contact surface side of the band cover 40 and in any portion of the surface on the opposite side to the contact surface side.

When attaching the band cover 40 to the band portion 20, for example, either end portion of the fixing band 21 is inserted from an opening of one end portion of the band cover 40 in the longitudinal direction, and the positions of each electrode 30 and the electrode opening 41 only need to be aligned. At this time, the main body housing 11 portion needs to be passed; however, the band cover 40 has elasticity, and thus the main body housing 11 portion can be passed without difficulty, and then the main body housing 11 can be exposed from the main body opening 42.

Here, the positional relationship between the electrode 30 and the electrode opening 41 when the band cover 40 is correctly attached to the band portion 20 will be described with reference to FIG. 3(A). As illustrated in FIG. 3(A), a state where the electrode opening 41 of the band cover 40 is covered by the protrusion portion 31 (more specifically, the umbrella portion 311) of the electrode 30 is a state where the band cover 40 is correctly attached. In this state, the fixing band 21 can be prevented from directly contacting the skin surface while the band cover 40 does not inhibit contact between the electrode 30 and the skin surface.

In addition, in order to cover the entire electrode opening 41 of the band cover 40 by the protrusion portion 31 of the electrode 30, for example, the band cover 40 may be attached in a state where the protrusion portion 31 of the electrode 30 is removed, and then the protrusion portion 31 may be fitted to the fitting portion 32 exposed from the electrode opening 41. Alternatively, even in a state where the protrusion portion 31 has already been fitted, the entire electrode opening 41 can be covered by the protrusion portion 31 by inserting the peripheral edge portion of the electrode opening 41 into a gap between the contact surface of the fixing band 21 and the bottom surface of the umbrella portion 311 of the protrusion portion 31 while stretching a portion located near the electrode opening 41 to expand the electrode opening 41.

### Functional Configuration

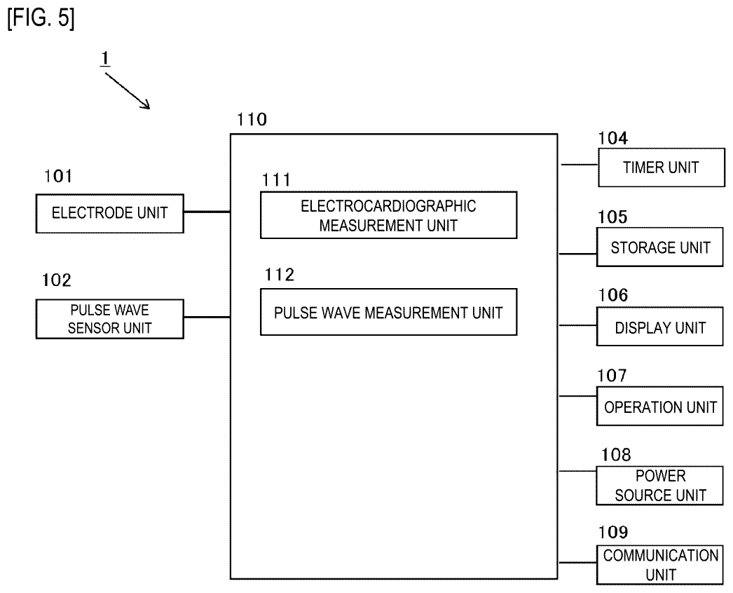

Next, a functional configuration of the biological information measurement device 1 will be described with reference to FIG. 5. FIG. 5 is a block diagram illustrating the functional configuration of the biological information measurement device 1. As illustrated in FIG. 5, the biological information measurement device 1 includes functional units of the control unit 110, an electrode unit 101, a pulse wave sensor unit 102, a timer unit 104, a storage unit 105, a display unit 106, an operation unit 107, a power source unit 108, and a communication unit 109.

The control unit 110 is means serving to control the biological information measurement device 1, and includes, for example, a central processing unit (CPU) and the like. Upon receiving operation of the user via the operation unit 107, the control unit 110 controls each component of the biological information measurement device 1 to execute various types of processing such as biological information measurement and information communication in accordance with a predetermined program. The predetermined programs are stored in the storage unit 105 which will be described later and read therefrom. In addition, the control unit 110 includes, as functional modules, an electrocardiographic measurement unit 111 and a pulse wave measurement unit 112.

The electrocardiographic measurement unit 111 acquires time series data of an electrocardiographic signal from the electrode unit 101, measures an electrocardiographic waveform of the user, and stores electrocardiogram (ECG) data in the storage unit 105. The pulse wave measurement unit 112 acquires time series data of a pulse wave signal from the pulse wave sensor unit 102, measures a pulse wave waveform of the user, and stores photoplethysmogram (PPG) data in the storage unit 105.

The electrode unit 101 includes the six electrodes 30a, 30b, 30c, 30d, 30e, and 30f and functions as a sensor unit configured to detect an electrocardiographic signal. Specifically, in a state where the biological information measurement device 1 is attached, two of the electrodes in an opposing positional relationship pair up with each other, and an electrocardiographic signal is detected based on a potential difference between the two electrodes in a pair. In other words, three types of electrocardiographic signals can be simultaneously detected from three pairs of electrodes.

The pulse wave sensor unit 102 includes a desired pulse wave sensor 14 and functions as a sensor unit configured to detect a pulse wave signal. The pulse wave sensor 14 in the present embodiment is a reflection type photoelectric pulse wave sensor disposed on the lower surface side of the main body housing 11 (i.e., the surface that comes int contact with the skin when worn) as illustrated in FIG. 2(B). The reflection type photoelectric pulse wave sensor can detect a blood flow volume (a change in volume of a blood vessel) that changes with a pulsation of the heart by irradiating a living body with infrared light, red light, or green light and detecting, with the use of a photodiode or the like, the light reflected in the living body.

Additionally, although not illustrated, each sensor unit including the electrode unit 101 includes an amplifier unit configured to amplify a signal output from the sensor, an A/D conversion unit configured to convert an analog signal into a digital signal, a filter circuit configured to remove a noise component, and the like.

The timer unit 104 functions to measure time with reference to a real time clock (RTC, not illustrated). For example, the time when a predetermined event occurs is counted and output.

The storage unit 105 includes a main storage (not illustrated) such as a random access memory (RAM) and stores various types of information such as application programs and biological information data measured by each measurement unit described below. In addition to the RAM, for example, a long-term storage medium such as a flash memory is provided, and thus long-term biological information can be stored.

The display unit 106 includes the LED indicator 12, and is configured to inform the user of a state of the device, occurrence of a predetermined event, and the like by lighting, blinking, or the like of the LED indicator 12. Additionally, the operation unit 107 includes a plurality of the operation buttons 13, and is configured to receive an input operation from the user via the operation button and function to cause the control unit 110 to execute processing in accordance with the operation.

The power source unit 108 includes a battery (not illustrated) configured to supply power required for operation of the device. The battery may be, for example, a secondary battery such as a lithium ion battery, or may be a primary battery. In a case where a secondary battery is provided, a charging terminal or the like may be provided. The communication unit 109 includes an antenna for wireless communication, a wire communication terminal (both not illustrated), and the like and functions to communicate with another device such as an information processing terminal. Note that the communication unit 109 may also serve as a charging terminal.

According to the biological information measurement device 1 of the present embodiment as described above, even when the electrode 30 is provided on the band portion 20, the surface of the fixing band 21 that comes in contact with the skin (excluding the electrode 30 portion) can be covered by the band cover 40, and thus cleanliness of the band portion 20 can be maintained. Further, since cleaning can be completed only by attaching and detaching the band cover 40, maintainability of the device is improved, and the operating time (measurement time of biological information) of the device can be increased.

Furthermore, a portion with which the skin surface comes in contact when the biological information measurement device 1 is worn is the band cover 40; therefore, by devising the material of the band cover 40, stuffiness or rash when the device is worn can be prevented. In other words, for the material of the fixing band 21, the need to consider constant contact with the living body can be eliminated. In addition, since the band cover 40 can be designed in various ways, the cover can be easily replaced in accordance with the time, place, and occasion.

Further, the electrode 30 of the band portion 20 is configured to include the protrusion portion 31 that comes in contact with the living body and the fitting portion 32 that is installed inside the fixing band 21, and the protrusion portion 31 is configured to be attachable to and detachable from the fitting portion 32. Therefore, the protrusion portion 31 can be easily replaced, and the maintainability of the device can be further improved. Furthermore, since the protrusion portion 31 is attachable and detachable, the band cover 40 can be easily attached and detached.

### Modified Examples

The structures of the electrode 30 and the fixing band 21, the arrangement relationship between the electrode opening 41 of the band cover 40 and the electrode 30, and the like can be configured in various ways other than the above-described embodiment. Such modified examples will be described below. Note that in the following modified examples, the same reference numerals are used for the same components as those of the biological information measurement device 1 according to the above-described embodiment, and the description thereof will be omitted. Only the differences from the above-described embodiment will be mainly described. In the schematic diagrams of the modified examples, components that do not need to be described are omitted as appropriate.

### First Modified Example

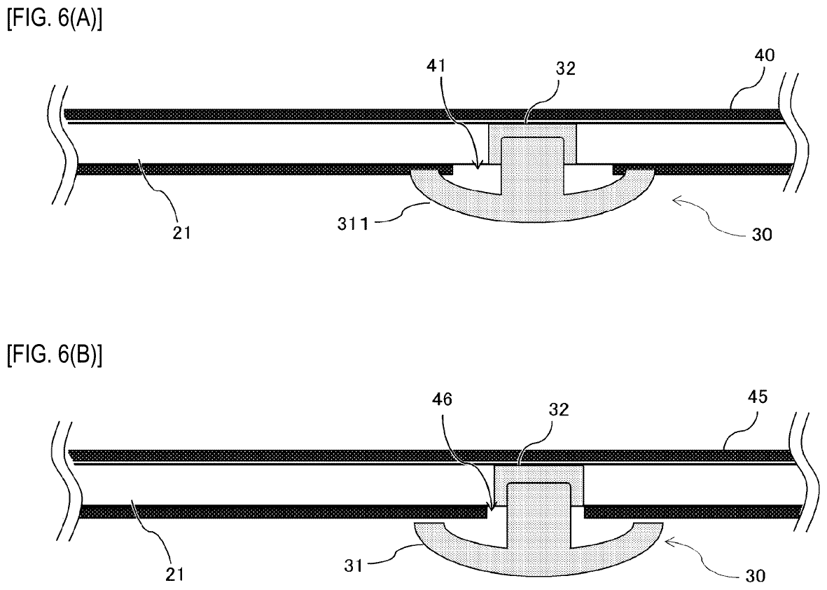

FIG. 6(A) is a schematic diagram illustrating configurations of the electrode 30 and the electrode opening 41 according to a first modified example. In the present modified example, in a state where the band cover 40 is attached as illustrated in FIG. 6(A), the peripheral edge portion of the electrode opening 41 is sandwiched between the protrusion portion 31 of the electrode 30 (more specifically, the end surface of the umbrella portion 311) and the contact surface of the fixing band 21. In other words, in a state where the protrusion portion 31 of the electrode 30 and the fitting portion 32 are fitted to each other, a gap between the contact surface of the fixing band 21 and the end surface of the umbrella portion 311 is configured to be smaller than the thickness of the band cover 40. According to such a configuration, the periphery of the electrode opening 41 is fixed, and the position of the band cover 40 can be prevented from being shifted.

### Second Modified Example

FIG. 6(B) is a schematic diagram illustrating the arrangement relationship between a mode of an electrode opening 46 of a band cover 45 and the electrode 30 according to a second modified example. In the present modified example, as illustrated in FIG. 6(B), the diameter of the electrode opening 46 of the band cover 45 is configured to be smaller than that of the embodiment. Thus, even when the electrode opening 46 is maximumly expanded, the electrode opening 46 is kept covered by the protrusion portion 31 of the electrode 30. According to such a configuration, even if the peripheral edge portion of the electrode opening 46 is not sandwiched between the protrusion portion 31 and the contact surface, the protrusion portion 31 of the electrode 30 can be prevented from being covered by the band cover 45 due to shifting of the position of the electrode opening 46.

### Third Modified Example

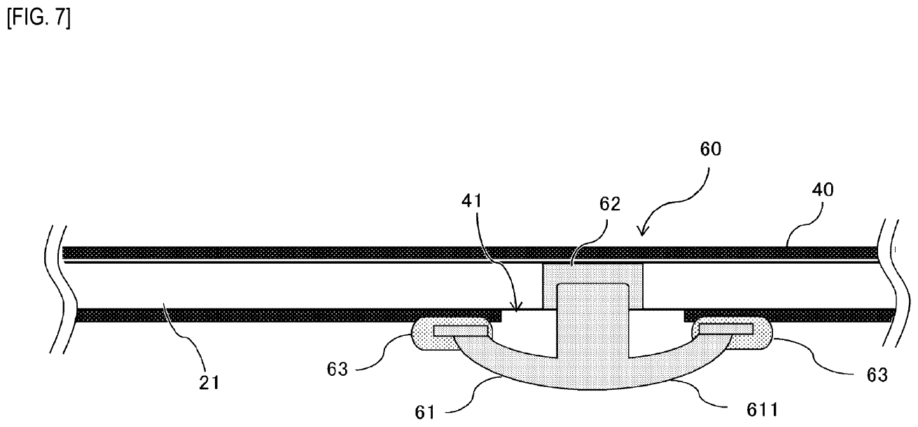

FIG. 7 is a schematic diagram illustrating a configuration of an electrode 60 according to a third modified example. The electrode 60 according to the present modified example is substantially the same as that of the first embodiment, and includes a protrusion portion 61 that protrudes in a dome shape from the contact surface of the fixing band 21 and a fitting portion 62 that is disposed inside the fixing band 21, and the protrusion portion 61 and the fitting portion 62 are configured to be detachably fitted to each other. However, the present modified example is different from the embodiment in that an elastic member 63 made of resin is provided at a base end portion (an end portion on the contact surface side) of the outer periphery of an umbrella portion 611 of the protrusion portion 61 of the electrode. With such a configuration, in a state where the band cover 40 is attached, movement of the peripheral edge portion of the electrode opening 41 is restricted by friction generated between the elastic member 63 and the band cover 40. According to such a configuration, even when the force of sandwiching the band cover 40 between the protrusion portion 61 of the electrode 60 and the contact surface of the fixing band 21 is weak, the position of the electrode opening 41 of the band cover 40 can be prevented from being shifted by frictional force of the elastic member 63.

### Fourth Modified Example

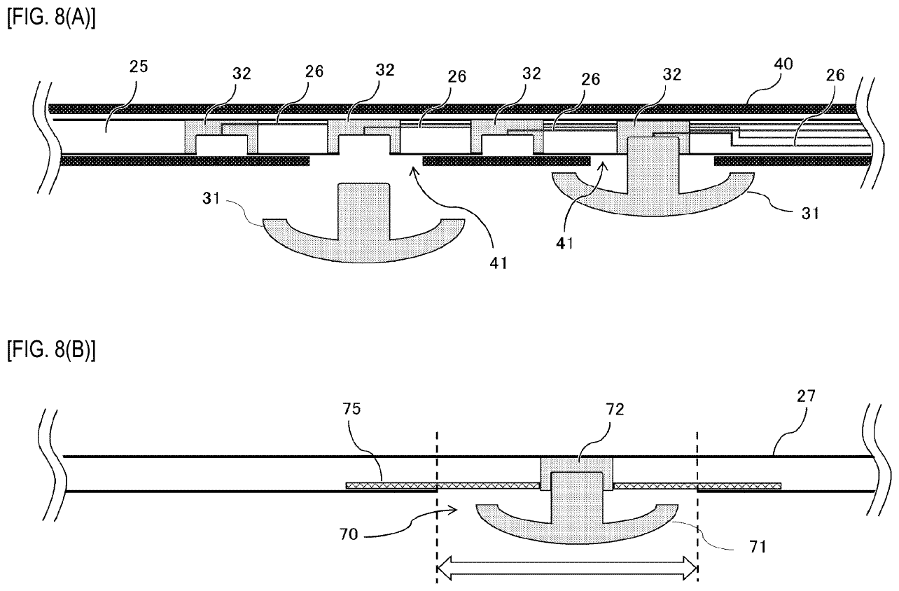

FIG. 8(A) is a schematic diagram illustrating a configuration of a fixing band 25 according to a fourth modified example. In the above-described embodiment, the number of fitting portions 32 provided in the fixing band 21 corresponds to the number of electrodes (six electrodes) 30. On the other hand, in the present modified example, six or more conductive fitting portions 32 that are fitted to the protrusion portions 31 of the electrodes 30 are provided in the fixing band 25, and thus the fitting portion 32 not fitted to the six protrusion portions 31 arises. In addition, in the fixing band 25, conductive wires (signal wires) 26 electrically connected to the main body housing 11 are connected to all the fitting portions 32, and each of the conductive wires 26 is disposed so as to avoid the fitting portions 32 other than the fitting portion 32 to which the conductive wire 26 itself is connected.

Since the protrusion portion 31 of the electrode 30 is configured to be able to be fitted to any of the fitting portions 32, the arrangement of the electrode 30 in the band portion can be changed depending on which fitting portion 32 the protrusion portion 31 is fitted into. In addition, the number of electrodes 30 functioning as a sensor unit can be changed by increasing or decreasing the number of protrusion portions 31.

With such a configuration, the arrangement and number of electrodes can be easily changed according to individual differences in the size of a site to which the device is attached (for example, the circumference of the arm), and thus the device having high convenience can be provided. In addition, as illustrated in FIG. 8(A), the band cover 40 in which after the arrangement of the electrode is determined, the position of the protrusion portion 31 and the position of the electrode opening 41 are matched is used, and thus the fitting portions 32 to which the protrusion portions 31 are not fitted can be covered by the band cover 40.

### Fifth Modified Example

FIG. 8(B) is a schematic diagram illustrating a configuration of a fixing band 27 and an electrode 70 according to a fifth modified example. In the present modified example, the electrode 70 includes a fitting portion 72 and a protrusion portion 71 that is configured to be attachable to and detachable from the fitting portion 72, which is the same as the above examples; however, the fitting portion 72 is configured to be slidable in the longitudinal direction inside the fixing band 27, and the arrangement of the electrode 70 can be changed by sliding the fitting portion 72. The open double-headed arrow in FIG. 8(B) indicates a range in which the fitting portion 72 can slide. Specifically, for example, a rail portion (not illustrated) is provided inside the fixing band 27, the fitting portion 72 is disposed on the rail, a large opening is provided on the contact surface side of the fixing band 27, and the entire region of the opening can be covered by a lid member 75 that moves with the fitting portion 72. In addition, in the present modified example, the band cover 40 in which after the electrodes 70 are positioned, the electrode openings 41 are provided at positions corresponding to the positions of the electrodes 70 only needs to be attached.

### Others

The descriptions of the above examples are merely illustrative of the present invention, and the present invention is not limited to the specific embodiments described above. In addition to the examples described above, various modifications and combinations may be made within the scope of the technical idea of the present invention. For example, in each of the examples described above, the electrode has a configuration in which the protrusion portion 31 and the fitting portion 32 are detachable, but the electrode does not necessarily need to be configured in this manner, and the protrusion portion does not need to be detachable. The band cover can be attached as long as there is a gap between the end surface of the protrusion portion and the contact surface of the fixing band into which the peripheral edge portion of the electrode opening of the band cover can be inserted. In addition, although the shape of the protrusion portion is configured to protrude in a dome shape in each of the examples described above, the protrusion portion does not necessarily need to be configured in this manner, and various shapes can be adopted as the shape of the protrusion portion (that is, the site that comes in contact with the skin surface) of the electrode.

Further, although the band cover is formed in a tubular shape in each of the examples described above, the band cover does not necessarily need to be formed in this manner, and can be formed as, for example, a seal having an adhesive surface. In other words, the band cover can be installed on the band portion by bonding the adhesive surface of the seal provided with the electrode openings to the contact surface of the fixing band. Even in such a configuration, the surface on the opposite side to the adhesive surface (that is, the surface that comes in contact with the skin surface when the band cover is attached) is formed of a cloth material, and thus discomfort when the device is attached can be suppressed.

Furthermore, in a case where the band cover is formed in a seal shape, the adhesive surface can be formed of a gel mat such as polyurethane. Thus, the band cover that has a seal shape and that can be washed with water and reused many times can be provided.

In addition, the biological information measurement device does not need to include a pulse wave sensor, and the present invention can also be applied to a measurement device that measures only an electrocardiographic waveform.

### REFERENCE SIGNS LIST

1 Biological information measurement device
10 Main body portion
11 Main body housing
12 LED indicator
13 Operation button
14 Pulse wave sensor
101 Electrode unit
102 Pulse wave sensor unit
104 Timer unit
105 Storage unit
106 Display unit
107 Operation unit
108 Power source unit
109 Communication unit
110 Control unit
20 Band portion
21, 25, 27 Fixing band
22 Band annular loop
23, 26 Conductive wire
30, 30a, 30b, 30c, 30d, 30e, 30f, 60, 70 Electrode
31, 61, 71 Protrusion portion
32, 62, 72 Fitting portion
40, 45 Band cover
41, 46 Electrode opening
42 Main body opening
63 Elastic member

1. A biological information measurement device to be used by being attached to a human body, the biological information measurement device comprising:
a main body housing;
a band portion in which a plurality of electrodes are disposed and which is used to attach the main body housing to the human body; and
a band cover that is detachable, formed of a non-conductive material, and configured to be able to cover at least a contact surface of the band portion on a side that comes in contact with the human body, wherein
the electrodes each include a protrusion portion provided to protrude from the contact surface of the band portion,
the band cover is formed of a material having elasticity and includes a plurality of opening portions, each of the opening portions being through which the protrusion portion of the electrode is provided to protrude,
the protrusion portion includes an umbrella portion formed in a dome shape and a shaft portion extending in a cylindrical shape from a central portion of the umbrella portion,
the opening portion has a diameter smaller than a diameter of the protrusion portion, and
the electrode and the opening portion are configured such that when covering the band portion by the band cover, the opening portion is expanded with the elasticity of the band cover to allow the umbrella portion to pass through the opening, and such that after the umbrella portion is passed through the opening portion, the opening portion is covered by the umbrella portion of the electrode.

2. The biological information measurement device according to claim 1, wherein the electrode is configured such that the protrusion portion is attachable to and detachable from the band portion.

3. The biological information measurement device according to claim 2, wherein when the band portion is covered by the band cover, the band cover is fixed by sandwiching at least a peripheral edge portion of the opening portion between the protrusion portion of the electrode and the contact surface.

4. The biological information measurement device according to claim 2, wherein
the band cover is formed of a material having elasticity, and
the electrode and the opening portion are configured such that the opening portion is covered by the protrusion portion of the electrode even when the opening portion is maximally expanded with the elasticity.

5. The biological information measurement device according to claim 2, wherein
the protrusion portion of the electrode is provided with an elastic member at a base end portion of an outer periphery in a plan view, and
when the band portion is covered by the band cover, the band cover is fixed by restricting movement of a peripheral edge portion of the opening portion by friction with the elastic member.

6. The biological information measurement device according to claim 2, wherein
the band portion is provided with a plurality of conductive fitting portions each fitted to the protrusion portion, and the protrusion portion of the electrode is attachable to and detachable from the fitting portion, and
a plurality of signal lines each electrically connecting a corresponding one of the fitting portions and the main body housing are disposed inside the band portion to avoid the other fitting portions.

7. The biological information measurement device according to claim 6, wherein at least the fitting portion has magnetic force, and the fitting portion and the protrusion portion are fixed by the magnetic force.

8. The biological information measurement device according to claim 6, wherein the protrusion portion of the electrode is configured to be attachable to and detachable from any of the plurality of fitting portions, and the arrangement and/or number of the electrodes is configured to be changeable by providing the fitting portion to which the protrusion portion is not fitted.

9. A biological information measurement device to be used by being attached to a human body, the biological information measurement device comprising:
a main body housing;
a band portion in which a plurality of electrodes are disposed and which is used to attach the main body housing to the human body; and
a band cover that is detachable, formed of a non-conductive material, and configured to be able to cover at least a contact surface of the band portion on a side that comes in contact with the human body, wherein
the electrodes each include a protrusion portion provided to protrude from the contact surface of the band portion,
the band cover includes a plurality of opening portions, each of the opening portions being through which the protrusion portion of the electrode is provided to protrude,
the electrode and the opening portion are configured such that the opening portion is covered by the protrusion portion of the electrode when the band portion is covered by the band cover,
the band portion is provided with a plurality of conductive fitting portions each fitted to the protrusion portion, and the protrusion portion of the electrode is attachable to and detachable from the fitting portion,
a plurality of signal lines each electrically connecting a corresponding one of the fitting portions and the main body housing are disposed inside the band portion to avoid the other fitting portions, and
the fitting portion is configured to be slidable, and the arrangement of the electrode is configured to be changeable by sliding the fitting portion.

10. The biological information measurement device according to claim 1, wherein the band cover is provided with an adhesive surface on a side that comes in contact with the contact surface.
